(19) United States
(12) Patent Application Publication
(10) Pub. No.: US 2009/0165797 A1
(43) Pub. Date: Jul. 2, 2009

(54) AIRWAY SYSTEM, OPERATING MEHTOD THEREOF AND VENTILATOR OR ANESTHETIC MACHINE HAVING SUCH SYSTEM

(75) Inventors: Yumeng CONG, Beijing (CN); Wei WANG, Beijing (CN); Hongyu Zhang, Beijing (CN)

Correspondence Address: WALKER & JOCKE, L.P.A., 231 SOUTH BROADWAY STREET, MEDINA, OH, 44256, US

(73) Assignee: BEIJING AEONMED CO., LTD., Beijing (CN)

(21) Appl. No.: 12/326,012

(22) Filed: Dec. 1, 2008

(30) Foreign Application Priority Data
Dec. 28, 2007 (CN) 200710306301.7

(51) Int. Cl. A61M 16/00

(52) U.S. Cl. 128/204.23

(57) ABSTRACT

The present invention discloses an airway system and operating method thereof in the medical equipments such as a ventilator and an anesthetic machine. The airway system comprises: a respiratory circuit (2) comprising an inspiratory branch (21) and an expiratory branch (22); a control unit (1) connected to the respiratory circuit (2) for controlling the opening and closing of the inspiratory and expiratory branches; an airway pressure sensor (7) located in the respiratory circuit (2) for measuring the airway pressure, wherein an ambient pressure sensor (5) is also located in the respiratory circuit (2), and wherein in the normal operating mode, the ambient pressure sensor (5) is used to measure the ambient pressure, while when failures occur in the airway pressure sensor (7) it is used to roughly measure the airway pressure. The airway system and operating method thereof according to the present invention guarantee the stable and continuous operation of the respiratory system when the airway pressure sensor can not measure the airway pressure because of failures so that the safety of the patient is ensured.

### TECHNICAL FIELD

The present invention relates to an airway system and operating method thereof, more specifically relates to an airway system and operating method thereof used in medical equipments such as ventilator, anesthetic machine and etc.

### BACKGROUND

In the usage of ventilator or anesthetic machine, airway pressure such as peak airway pressure, mean airway pressure, inspiratory pause pressure (plateau pressure), expiratory end pressure and etc may be measured. Moreover, as the airway pressure is continuously changing in each respiratory period, airway pressure sensors are required to monitor and measure the airway pressure in real time to prevent the airway pressure from being too high or too low and to maintain effective flow passing, so that the safety of the patient is guaranteed and serious complications are prevented.

At present, in anesthetic machines or ventilators, generally ambient pressure sensor is mounted outside the airway system, while airway pressure sensor is positioned inside the airway system. Using absolute pressure, the measuring range of the ambient pressure sensor is broader (generally 0-400 kPa). However, using relative pressure (relative to the atmospheric pressure), that of the airway pressure sensor is narrower (the range of the relative measuring pressure is −2-10 kPa). Compared with the ambient pressure sensor mounted outside the airway system, the sensor located in the internal airway system for measuring airway pressure is usually a precision pressure sensor. When failures occur in this kind of pressure sensor, the condition of airway pressure can not be determined, which may endanger the patient.

### SUMMARY OF THE INVENTION

The present invention is provided to solve the above problem and the object of the present invention is that, the ambient pressure sensor located in the air circuit is used to roughly measure the airway pressure when failures occur in the airway pressure sensor, so as to maintain the continuous and regular function of the system and to guarantee the safety of the patient.

According to one aspect of the present invention, there provides an airway system comprising: a respiratory circuit, which comprises an inspiratory branch one end of which is connected to driving gas via an inspiratory valve and the other end of which is connected to an inspiratory end of a terminal and an expiratory branch one end of which is connected to an expiratory end of the terminal and the other end of which is connected to an atmospheric end via a PEEP (positive end expiratory pressure) valve; a control unit connected to the respiratory circuit for controlling the opening and closing of the inspiratory valve and the PEEP valve; and an airway pressure sensor located in the respiratory circuit for measuring the airway pressure, wherein in the respiratory circuit also provides an ambient pressure sensor, which in a first operating mode measures the ambient pressure by closing the inspiratory valve and completely opening the PEEP valve, while in a second operating mode measures airway pressure by opening the inspiratory valve and closing the PEEP valve or opening the PEEP valve and closing the inspiratory valve.

Preferably, in the first operating mode, the ambient pressure sensor measures the ambient pressure by closing the inspiratory valve and completely opening the PEEP valve, while in the second operating mode it measures the airway pressure by opening the inspiratory valve and closing the PEEP valve or opening the PEEP valve partly and closing the inspiratory valve.

Preferably, the first operating mode is the normal operating mode, while the second operating mode is the operating mode when failures occur in the airway pressure sensor.

Preferably, the ambient pressure sensor and the airway pressure sensor are connected to the control unit via a respective data acquiring unit.

Preferably, in view of the factors of structure design, the ambient pressure sensor is located in the inspiratory branch, while the airway pressure sensor is located in the expiratory branch.

Preferably, the operating range of the ambient pressure sensor is 0-400 kPa.

According to another aspect of the present invention, there provides a ventilator, which includes an airway system comprising a respiratory circuit, which comprises an inspiratory branch one end of which is connected to driving gas via an inspiratory valve and the other end of which is connected to an inspiratory end of a terminal and an expiratory branch one end of which is connected to an expiratory end of the terminal and the other end of which is connected to an atmospheric end via PEEP valve; a control unit connected to the respiratory circuit for controlling the opening and closing of the inspiratory valve and the PEEP valve; and an airway pressure sensor located in the respiratory circuit for measuring the airway pressure, wherein in the respiratory circuit also provides an ambient pressure sensor, which in a first operating mode measures the ambient pressure by closing the inspiratory valve and completely opening the PEEP valve, while in a second operating mode measures the airway pressure by opening the inspiratory valve and closing the PEEP valve or opening the PEEP valve and closing the inspiratory valve.

According to yet another aspect of the present invention, there provides an anesthetic machine, which includes an airway system comprising a respiratory circuit, which comprises an inspiratory branch one end of which is connected to driving gas via an inspiratory valve and the other end of which is connected to an inspiratory end of a terminal and an expiratory branch one end of which is connected to an expiratory end of the terminal and the other end of which is connected to an atmospheric end via PEEP valve; a control unit connected to the respiratory circuit for controlling the opening and closing of the inspiratory valve and the PEEP valve; and an airway pressure sensor located in the respiratory circuit for measuring the airway pressure, wherein in the respiratory circuit also provides an ambient pressure sensor, which in a first operating mode measures the ambient pressure by closing the inspiratory valve and completely opening the PEEP valve, while in a second operating mode measures the airway pressure by opening the inspiratory valve and closing the PEEP valve or opening the PEEP valve and closing the inspiratory valve.

According to still another aspect of the present invention, there provides an operating method of an respiratory system comprising the following steps: the inspiratory valve is closed and the PEEP valve is completely opened by the control unit and the ambient pressure sensor is used to measure the ambient pressure; the control unit is used to control the opening and closing of the inspiratory valve and the PEEP valve, so that the corresponding inspiratory and expiratory phases are entered and the airway pressure sensor is used to measure airway pressure accordingly; when failures occur in the airway pressure sensor, the control unit is used to control the opening and closing of the inspiratory valve and the PEEP valve and the ambient pressure sensor is used to measure the airway pressure.

Wherein the step of measuring the airway pressure by the ambient pressure sensor in real time comprises that in the inspiratory phase, the inspiratory valve is opened and the PEEP valve is completely closed via the control unit and the ambient pressure sensor measures the airway pressure; and that in the expiratory phase, the PEEP valve is partly opened and the inspiratory valve is closed and the ambient pressure sensor measures the airway pressure.

According to the technical solution of the present invention, the ambient pressure sensor is located inside the airway system, wherein the ambient pressure can be measured when the PEEP valve is completely opened and the inspiratory valve is closed; and the airway pressure can be measured when the PEEP valve is closed and the inspiratory valve is opened or when the PEEP valve is partly opened and inspiratory valve is closed. In this way, one sensor serves two functions so that the ambient pressure sensor can be used to roughly measure and measure the airway pressure when failures occur in the airway pressure sensor, which enhances the practicability and safety of the apparatus, protect the terminal (such as patients) and avoid dangerous accidents.

### BRIEF DESCRIPTION OF THE DRAWINGS

The above and other aspects, features and advantages may be better understood with reference to the following detailed description in conjunction with the figures, in which:

FIG. 1 is a block diagram illustrating the structure of the airway system used in a ventilator according to an embodiment of the present invention;

FIG. 2 is a flow chart showing the operating method of the airway system used in a ventilator according to the embodiment of the present invention.

### DETAILED DESCRIPTION OF THE PREFERRED EMBODIMENTS

The embodiment of the present invention will be further described in details with reference to the accompanying figures as follows. However, the present invention can be carried out in various forms and should not be limited to the embodiments illustrated hereafter. On the contrary, the embodiments here are intended to the present disclosure complete and comprehensive and provide a complete scope of protection of the present invention to those skilled in the art.

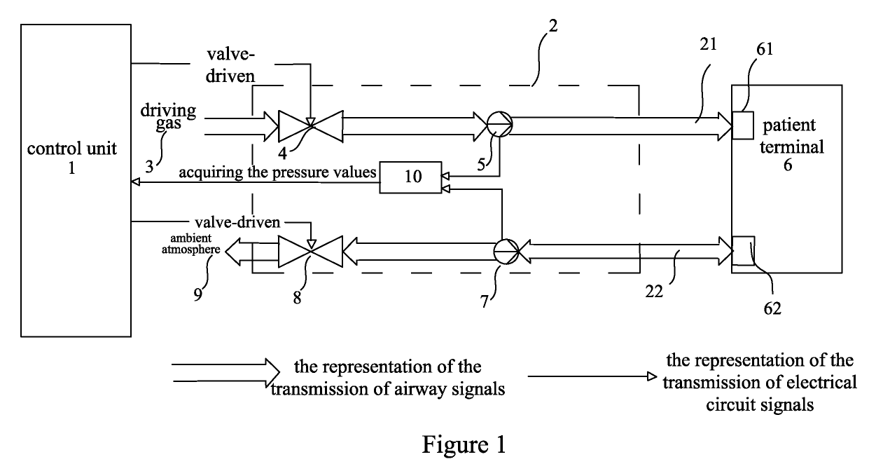

FIG. 1 is a block diagram illustrating the structure of the airway system used in a ventilator according to the embodiment of the present invention. As shown in FIG. 1, the airway system of this embodiment comprises a control unit 1 and a respiratory circuit 2 connected to the control unit 1. The respiratory circuit 2 comprises an inspiratory branch 21 and an expiratory branch 22. Driving gas 3 flows into the inspiratory branch 21 through an inspiratory valve 4 and finally into an inspiratory port 61 of the patient terminal 6. The gas exhausted from an expiratory port 62 of the patient terminal 6 flows into the expiratory branch 22 and is discharged into the ambient port 9 through PEEP valve 8. In the expiratory branch 22, there is provided an airway pressure sensor 7 for measuring the airway pressure (whose relative pressure is −2-10 kPa) of the patient in the respiratory phase and the values of the airway pressure are transmitted to the control unit 1 through a data acquiring unit 10. The efficient flow of the ventilator is guaranteed and serious complications are prevented by virtue of the measuring of the airway pressure in real time. In the airway system of this embodiment, there is also provided an ambient pressure sensor 5 in the inspiratory branch 21 between the inspiratory valve 4 and the inspiratory port 61, by which the value of the ambient pressure measured is transmitted to the control unit 1 via the data acquiring unit 10.

In the normal operating conditions, before the system operates, the control unit 1 closes the inspiratory valve 4 and completely opens the PEEP valve 8 so that the ambient pressure sensor 5 can function as an ambient pressure sensor located outside the ventilator in a traditional ventilator does, i.e. to measure the pressure of the operating environment, so as to set and regulate the relative parameters of the ventilator based on the measured pressure. According to the present invention, however, the ambient pressure sensor 5 is located inside the airway system, which has the advantage that the ambient pressure sensor 5, as located in the airway system, can provisionally and roughly measure the airway pressure when the airway pressure sensor 7 is unable to measure because of failures, so as to guarantee the stable and continuous operation of the ventilator and the safety of the patient.

According to the present invention, the operating principle of the airway system is as follows:

In the inspiratory phase, the inspiratory valve is opened and the PEEP valve is closed. At this time the pressure in the respiratory circuit is the airway pressure; as the ambient pressure sensor is located in the airway, the condition of the airway pressure can be monitored and measured.

When the inspiratory phase ends and the expiratory phase of the system begins, the inspiratory valve is closed and the PEEP valve is partly opened; as the pressure in the airway is larger than the external atmospheric pressure, the pressure measured by the ambient pressure sensor is the airway pressure in the expiratory phase.

Therefore, the ambient pressure sensor can measure both the airway pressure and the ambient pressure, that is, one sensor serves two functions, which guarantees the stable and continuous operation of the system and the safety of the patient.

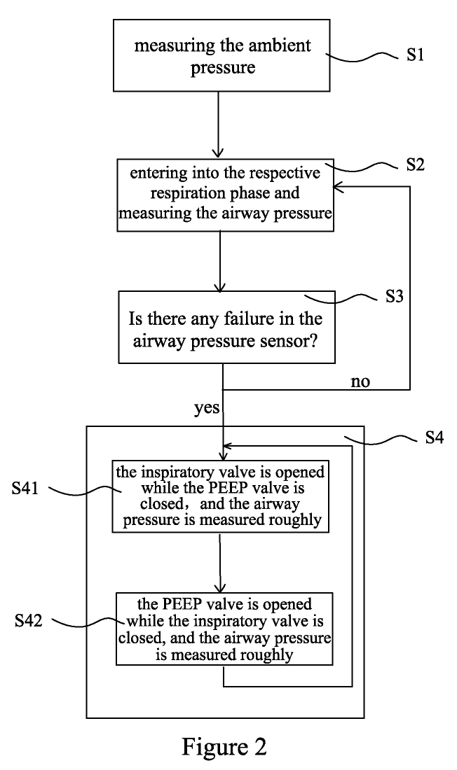

Based on the above operating principle, FIG. 2 is a flow chart showing the operating method of the airway system used in a ventilator according to the embodiment of the present invention.

As shown in FIG. 2, in step S1, the inspiratory valve 4 is closed and the PEEP valve 8 completely opened by the control unit 1, the ambient pressure sensor 5 is used to measure the ambient pressure and the value of the ambient pressure is transmitted to the control unit 1 via the data acquiring unit 10. Subsequently, in step S2, the control unit 1 sets and regulates the operating parameters based on the obtained ambient pressure value and makes the airway system to enter the respective inspiratory phase (during which the inspiratory valve is opened, while the PEEP valve is closed) or expiratory phase (during which the inspiratory valve is closed, while the PEEP valve is partly opened) by controlling the opening and closing of the inspiratory valve 4 and the PEEP valve 8 and the data acquiring unit 10 transmits the value of the pressure (i.e. value of the airway pressure) measured by the airway pressure sensor 7 to the control unit 1.

When the airway pressure sensor can not measure the airway pressure because of failures (S3), as shown in step S4, in order to guarantee the safety of the patient and the stable and continuous operation of the system, at this time the ambient pressure sensor 5 is used to roughly measure the status of the airway pressure, wherein:

In the inspiratory phase, as shown in step S41, the inspiratory valve 2 is opened and the PEEP valve 8 is closed. As located in the airway, the ambient pressure sensor 5 can roughly measure the status of the airway pressure. When the inspiratory phase ends and the expiratory phase begins, as shown in step S42, the PEEP valve 8 is opened, the inspiratory valve 4 is closed, and the driving gas in the expiratory branch is discharged into the external atmosphere through the partly opened PEEP valve 8. As the PEEP valve is partly opened in the expiratory phase, the airway pressure is larger than the external atmosphere and therefore the pressure measured by the ambient pressure sensor 5 is the airway pressure at that time.

With different properties, the ambient pressure sensor can not measure the value of the airway pressure very exactly, it can only measure the status of the airway pressure roughly to guarantee the continuous operation of the system without interruption and to avoid dangers to the patient.

In order to better show the status of the airway pressure, preferably, the operating range of the ambient pressure sensor is 0-400 kPa.

The present invention is described above with reference to the embodiment which is considered to be practicable at present, however, it can be understood by those skilled in the art that the present invention is not limited to the disclosed embodiment here given as an example. Any amendments, equivalent substitutions, improvements and etc, within the spirit and principle of the present invention, should be considered to be covered in the scope of protection of the present invention.

What is claimed:

1. An airway system comprising:
a respiratory circuit (2), which comprises
an inspiratory branch (21), one end of which is connected to driving gas (3) via an inspiratory valve (4) and the other end of which is connected to an inspiratory port (61) of a terminal (6); and
an expiratory branch (22), one end of which is connected to an expiratory port (62) of the terminal (6) and the other end of which is connected to an ambient port (9) via a PEEP valve (8);
a control unit (1), connected to the respiratory circuit (2) for controlling the opening and closing of the inspiratory valve (4) and the PEEP valve (8); and
an airway pressure sensor (7), located in the respiratory circuit (2) for measuring the airway pressure;
characterized in that, in the respiratory circuit (2) there is also provided an ambient pressure sensor (5), which in a first operating mode measures the ambient pressure by closing the inspiratory valve (4) and opening the PEEP valve (8), while in a second operating mode measures the airway pressure by opening the inspiratory valve (4) and closing the PEEP valve (8) or by opening the PEEP valve (8) and closing the inspiratory valve (4).

2. The airway system according to claim 1, characterized in that, in the first operating mode, the ambient pressure sensor (5) measures the ambient pressure by closing the inspiratory valve (4) and completely opening the PEEP valve (8), while in the second operating mode it measures the airway pressure by opening the inspiratory valve (4) and closing the PEEP valve (8) or by opening the PEEP valve (8) partly and closing the inspiratory valve (4).

3. The airway system according to claim 1, characterized in that, the first operating mode is the normal operating mode, while the second operating mode is the operating mode when failures occur in the airway pressure sensor (7).

4. The airway system according to claim 1, characterized in that, the ambient pressure sensor (5) and the airway pressure sensor (7) are connected to the control unit (1) respectively via a data acquiring unit (10).

5. The airway system according to in claim 1, characterized in that, the ambient pressure sensor (5) is located in the inspiratory branch (21), while the airway pressure sensor (7) is located in the expiratory branch (22).

6. The airway system according to in claim 1 or 2, characterized in that, the operating range of the ambient pressure sensor (5) is 0-400 kPa.

7. A ventilator, characterized in that, it comprises the airway system according to any one of claims 1 to 5.

8. An anesthetic machine, characterized in that, it comprises the airway system according to any one of claims 1 to 5.

9. An operating method of the airway system according to claim 1, comprising the following steps:
the inspiratory valve (4) is closed and the PEEP valve (8) is completely opened by the control unit (1) and the ambient pressure sensor (5) is used to measure the ambient pressure;
the control unit (1) is used to control the opening and closing of the inspiratory valve (4) and the PEEP valve (8) so that the corresponding inspiratory and expiratory phases are entered and the airway pressure sensor (7) is used to measure the airway pressure; and
when failures occur in the airway pressure sensor (7), the control unit (1) is used to control the opening and closing of the inspiratory valve (4) and the PEEP valve (8) and the ambient pressure sensor (5) is used to measure the airway pressure.

10. The operating method according to claim 9, characterized in that, the step of measuring the airway pressure by the ambient pressure sensor (5) in real time comprises:
in the inspiratory phase, the inspiratory valve (4) is opened and the PEEP valve (8) is completely closed by the control unit (1) and the ambient pressure sensor (5) measures the airway pressure; and
in the expiratory phase, the PEEP valve (8) is partly opened and the inspiratory valve (4) closed and the ambient pressure sensor (5) measures the airway pressure.
